(19) United States
(12) Patent Application Publication
(10) Pub. No.: US 2007/0276426 A1
(43) Pub. Date: Nov. 29, 2007

(54) Steerable balloon catheters and methods

(76) Inventor: Charles Louis Euteneuer, St. Michael, MN (US)

Correspondence Address: CYR & ASSOCIATES, P.A., 605 U.S. Highway 169, Suite 300, Plymouth, MN, 55441, US

(21) Appl. No.: 11/442,088

(22) Filed: May 26, 2006

(51) Int. Cl. A61M 5/178; A61M 29/00

(52) U.S. Cl. 606/192; 604/164.13

(57) ABSTRACT

Steerable balloon apparatus to access bodily lumen of a patient are disclosed. The steerable balloon apparatus include an inflation tube, an extendable member and a balloon. The extendable member is secured to a distal end of the balloon and to the distal end of the inflation tube. The extendable member can be positioned within an inflation chamber of the balloon. The balloon can be secured over distal end of the inflation tube. The steerable balloon apparatus can also include a core wire. The balloon can be slidably secured over the portion of the core wire. A proximal tube can be provided at the proximal end of the inflation tube.

### BACKGROUND OF THE INVENTION

1. Summary of the Invention

The present inventions relate to medical devices and, more particularly, to medical catheters and medical guidewires for insertion into bodily lumen of patients.

2. Description of the Related Art

Medical catheters and guidewires can be useful tools in treating intravascular disorders, disorders within other lumen of the body, extracting fluids from lumen as well as introducing fluid into lumen. Some medical catheters and most guidewires are configured to be received through a medical device to permit the medical device to be slid over the medical catheter or guidewire and positioned within the body of a patient. Further, many catheters and some guidewire designs include a balloon at or near the distal end of the catheter or guidewire. Depending on the configuration, these devices can also be used to introduce and/or expand various other medical devices, such as stents for example. The balloons may help direct the distal end of the catheter through a lumen where the pulsatile flow of blood may permit them to act as a “sail.” Further, the balloons in various configurations may be used to test for the occlusion of vessels, for embolization for bleeding, to treat or control vasospasms, and for treatment of nosebleeds, among other uses.

Medical catheters and guidewires are particularly useful in accessing remote and tortuous locations within the body. Because of the need to navigate through the body to remote locations through narrow twisting lumen, medical catheters and guidewires are frequently long thin devices. Frequently, the procedures using medical catheters and guidewires are time sensitive. Accordingly, these devices typically need to be easily guidable in an efficient manner by a user.

These devices frequently are subjected to various localized forces as they are torqued and pushed into position by a surgeon and the devices come into contact with various bodily structures within a patient. During positioning procedures, some configurations of the balloons positioned distally on the catheter or guidewire can collapse, bend, twist or otherwise deform. This twisting can form creases in the balloon that can catch or otherwise impede the navigation of the bodily lumen. This can inhibit or slow the surgeon's placement of the balloon at a desired location in the patient which in certain circumstances can be deleterious to a patient. The deformation of the balloon during implantation may also prevent or alter the balloons ability to be expanded after the surgeon has properly positioned the balloon which again can be deleterious to a patient. The deformation of the balloon after inflation can prevent or alter the balloons ability to be deflated as a surgeon is removing or preparing to remove the medical catheter and guidewire from the patient. Among other problems, the ability to deflate the balloon can complicate or prevent the ability to remove the medical catheter and guidewire from the patient. Accordingly, a need exists for balloon configurations that provide desired performance characteristics while being resistant to deformation during positioning procedures.

### SUMMARY OF THE INVENTION

Apparatus and methods in accordance with the present invention may resolve many of the needs and shortcomings discussed above and will provide additional improvements and advantages as will be recognized by those skilled in the art upon review of the present disclosure.

The present inventions provide steerable balloon apparatus for accessing a target location in a bodily lumen of a patient. A steerable balloon apparatus in accordance with one or more of the present inventions may include an inflation tube, a balloon and an extendable member. In one aspect, the steerable balloon apparatus may further include a core wire extending through the inflation tube. In another aspect, the steerable balloon apparatus may further include a proximal tube. The proximal tube may define a proximal lumen. The proximal lumen may extend between a proximal end and a distal end of the proximal tube. The distal end of the proximal tube may be secured to a proximal end of the inflation tube with the proximal lumen of the proximal tube in fluid communication with the inflation tube lumen of the inflation tube.

The inflation tube generally has an outer surface and an inner surface. The inner surface of the inflation tube defines an inflation tube lumen. The inflation tube lumen can extend along at least a portion of the inflation tube. The inflation tube further defines a distal inflation tube opening in communication with the inflation tube lumen. When present, the proximal tube may define a proximal lumen. The proximal tube lumen may extend between a proximal end and a distal end of the proximal tube. The distal end of the proximal tube may be secured to a proximal end of the inflation tube. When secured to the inflation tube, the proximal lumen of the proximal tube may be in fluid communication with the inflation tube lumen of the inflation tube. In one aspect, the distal end of the proximal tube may define a proximal tube notch. The proximal tube notch may receive a proximal end of the core wire extending from a proximal end of the inflation tube. The proximal end of the core wire may be secured within the proximal tube notch of the proximal tube. The core wire may define a longitudinal core wire mating surface and/or a perpendicular core wire mating surface. The longitudinal notch surface defining at least a portion of the notch may be secured to the longitudinal core wire mating surface. The perpendicular core wire mating surface of the core wire may be secured to a perpendicular notch surface of the proximal tube.

The balloon may be secured over a distal portion of the inflation tube. The balloon defines an inflation chamber in an un-inflated configuration or an at least partially inflated configuration. The inflation chamber is typically in fluid communication with distal inflation tube opening. When a core wire is provided, the core wire may extend through the inflation chamber of the balloon. In one aspect, the balloon may include a sleeve defining a sleeve passage. The sleeve may be integral with the balloon or may be a separate component secured to the balloon. When a core wire is provided, the sleeve may slidably receive the core wire through the sleeve passage. In one aspect, the sleeve comprising an end cap peripherally secured to the balloon.

The extendable member is generally configured to be extendable between at least a first length and a second length. The extendable member has a proximal end and a distal end. The proximal end of the extendable member may be secured to a distal end of the inflation tube. Typically, at least a portion of the extendable member is positioned within the inflation chamber of the balloon. The distal end of the extendable member may be secured to a distal end of the balloon. The extendable member may be secured with the distal lumen opening of the lumen. In one aspect, the extendable member defines at least one fenestration. The extendable member may comprise a coil.

Other features and advantages of the invention will become apparent from the following detailed description, and from the claims.

### BRIEF DESCRIPTION OF THE DRAWINGS

FIG. 1 illustrates a perspective view of an exemplary embodiment of a steerable balloon apparatus in accordance with the present inventions;

FIG. 2 illustrates a partial cross-sectional side view of an exemplary embodiment of an interconnection of a proximal tube and an inflation tube in accordance with the present inventions;

FIG. 3 illustrates a partial perspective view of an exemplary embodiment of a distal portion of a proximal tube including a notch in accordance with the present inventions;

FIG. 4 illustrates a partial perspective view of an exemplary embodiment of a proximal portion of a core wire in accordance with the present inventions;

FIG. 5A illustrates a transverse cross-section through section lines A-A of the embodiment of the interconnection illustrated in FIG. 2;

FIG. 5B illustrates a transverse cross-section through section lines B-B of the embodiment of the interconnection illustrated in FIG. 2;

FIG. 5C illustrates a transverse cross-section through section lines C-C of the embodiment of the interconnection illustrated in FIG. 2;

FIG. 6 illustrates a detailed partial perspective view of an embodiment of a distal portion of a steerable balloon apparatus in accordance with the present inventions;

FIG. 7A illustrates a cross-section of a partial side view of another exemplary embodiment of a distal end of a steerable balloon apparatus with an un-inflated balloon in accordance with the present inventions;

FIG. 7B illustrates a cross-section of a partial side view of an embodiment of a distal end of a steerable balloon apparatus similar to the embodiment of FIG. 7A with a partially inflated balloon in accordance with the present inventions;

FIG. 7C illustrates a cross-section of a partial side view of an embodiment of a distal end of a steerable balloon apparatus similar to the embodiment of FIG. 7A and 7B with a substantially fully inflated balloon in accordance with the present inventions;

FIG. 8A illustrates a cross-section of a partial side view of another exemplary embodiment of a distal end of a steerable balloon apparatus with an un-inflated balloon in accordance with the present inventions; and

FIG. 8B illustrates a cross-section of a partial side view of an embodiment of a distal end of a steerable balloon apparatus similar to the embodiment of FIG. 8A with an at least partially inflated balloon in accordance with the present inventions.

All Figures are illustrated for ease of explanation of the basic teachings of the present invention only; the extensions of the Figures with respect to number, position, relationship and dimensions of the parts to form the embodiment will be explained or will be within the skill of the art after the following description has been read and understood. Further, the exact dimensions and dimensional proportions to conform to specific force, weight, strength, flow and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings and utilized only to facilitate describing the illustrated embodiments. Similarly, when the terms “proximal,” “distal,” and similar positional terms are used, the terms should be understood to reference the structures shown in the drawings as they will typically be utilized by a physician or other user who is treating or examining a patient with apparatus in accordance with the present inventions.

### DETAILED DESCRIPTION OF THE INVENTION

### —General Overview of Steerable Balloon Apparatus

The present inventions provide steerable balloon apparatus 10 and associated methods for use in conjunction with medical catheters and medical guidewires. The figures generally illustrate embodiments of steerable balloon apparatus 10 including aspects of the present inventions. The particular exemplary embodiments of the steerable balloon apparatus 10 illustrated in the figures have been chosen for ease of explanation and understanding of various aspects of the present inventions. These illustrated embodiments are not meant to limit the scope of coverage but instead to assist in understanding the context of the language used in this specification and the appended claims. Accordingly, variations of steerable balloon apparatus 10 for use with medical guidewires and medical catheters different from the illustrated embodiments may be encompassed by the appended claims.

Steerable balloon apparatus 10 are generally configured to be introduced into a bodily lumen of a patient and have the balloon 18 positioned at a target location in the bodily lumen. The balloon 18 may then be inflated for a wide variety of medical purposes. Typically, the balloon 18 is inflated with an inflation media. The inflation media will frequently include various imaging compounds and may include various medicinal or other compounds that can be desirable in particular applications.

For purposes of this description, steerable balloon apparatus 10 generally should be considered to have longitudinal axis 300 defined along its length as generally illustrated in the Figures regardless of any curvature in the steerable balloon apparatus 10.

The steerable balloon apparatus 10 in accordance with the present inventions includes at least an inflation tube 14, an extendable member 16 and a balloon 18. The balloon 18 is secured to an end of the inflation tube 14 to permit the introduction of inflation media through the inflation tube 14 and into the balloon 18. A proximal portion of the extendable member 16 is secured to distal end of the inflation tube 14. A distal portion of the extendable member extends from the distal end of the inflation tube. Typically, the balloon 18 is secured over the extendable member 16 such that at least a portion of the extendable member 16 is positioned within the inflation chamber 28 of the balloon 18. A distal portion of the extendable member 16 is secured to the balloon 18. As the balloon is expanded with inflation media, the extendable member extends along the longitudinal axis of the balloon 18 to permit the distal end of the balloon to elongate in at least along its longitudinal axis. In operation, the extendable member 16 and the balloon 18 generally cooperate to reduce twisting and other deformation of the balloon 18 as the balloon 18 is positioned within a patient.

In addition, the steerable balloon apparatus 10 may include a core wire 20. The core wire 20 can be positioned through the inflation tube 14 and extendable member 16. The core wire 20 may confer desired performance characteristics such as a desired torquability or a desired pushability to the steerable balloon apparatus 10. In one aspect, the core wire 20 extends distally from the distal end of balloon 18 through a sleeve 38. The sleeve 38 may slidably receive the core wire 20 to permit the sliding of the distal end of balloon 18 along the longitudinal axis of core wire 20 during inflation and/or deflation of balloon 18. The sleeve 38 may also form a seal about the core wire 20. The seal is typically configured to prevent medically significant amounts of inflation media from leaking from between the core wire 20 and the sleeve 38.

As generally illustrated throughout the Figures for exemplary purposes, steerable balloon apparatus 10 includes at least an inflation tube 14, an extendable member 16 and a balloon 18. As illustrated, the steerable balloon apparatus 10 may further include a proximal tube 12. A distal end 212 of the proximal tube 12 may be secured to a proximal end 114 of the inflation tube 14 to permit the transmission of guiding forces and/or communications of fluids between the proximal tube 12 and the inflation tube 14. To secure the inflation tube 14 to the proximal tube 12, the proximal tube 12 may define a proximal tube notch 52 to receive a core wire 20 extending from the inflation tube 14. For purposes of the present inventions, the term “secured to” means that the distal tubular portion is attached to main proximal portion by a suitable method such as, for example, by welding, brazing, heat shrinking, or gluing among other methods. To communicate fluids, an inner surface 72 of the proximal tube 12 may define a proximal lumen 22. The inner surface 74 of the inflation tube 14 may define at least one inflation tube lumen 24. The distal opening 42 of the proximal lumen 22 may be secured in fluid communication with a proximal inflation opening 34 of the inflation tube lumen 24 when the distal end 212 of the proximal tube 12 is secured to a proximal end 114 of the inflation tube 14. A bridge tube 30 may be provided between the proximal lumen 22 and inflation tube lumen 24. At the distal end 214 of the inflation tube 14, the balloon 18 can be secured over an outer surface 64 of the inflation tube 14. The inflation chamber 28 of balloon 18 is typically defined, at least in part, by an inner surface 78 of the balloon 18. The inflation chamber 28 can be in fluid communication with a distal inflation opening 44 of the inflation tube lumen 24. Typically, the distal inflation opening 44 defined in distal end 214 of the inflation tube 14 communicates fluid from the inflation tube lumen 24 into the inflation chamber 28 of the balloon 18. In various aspects, the distal inflation tube opening 44 may be located at or near the distal end 214 of the inflation tube 14. A proximal inflation opening 34 in fluid communication with the inflation tube lumen 24 may be located at or near the proximal end 114 of the inflation tube 14 for the introduction of inflation media from a location remote from the balloon 18. The extendable member 16 can be secured to and extend from the distal end 214 of the inflation tube 14. In one aspect, the extendable member 16 may be in the form of a coiled wire. Typically, a proximal end 116 of the extendable member 16 is secured about the distal inflation opening 44 of the inflation tube lumen 24. The extendable member 16 may generally extend from the inflation tube 14 in an orientation parallel or coaxial with the longitudinal axis 300 of the inflation tube 14. The extendable member 16 typically extends into the inflation chamber 28 of the balloon 18. The distal end 218 of balloon 18 is secured to at least a portion of the extendable member 16. In one aspect, the extendable member 16 is secured to a sleeve 38 which is secured to a distal end 218 of balloon 18. The sleeve 38 may be integral with the balloon 18 or a separate structure secured to the balloon 18. The extendable member 16 is typically to extend longitudinally as the distal end 218 of the balloon 18 extends longitudinally during inflation and deflation of the balloon 18. A stop 36 may be provided at or near the distal end of the core wire 20 to prevent the distal end of the balloon 18 from extending beyond the end of the extendable member 16. When present, the core wire 20 may be received within the lumen 30. The core wire 20 may extend from the lumen 30 through distal inflation opening 44 of the inflation tube lumen 24. In one aspect, at least a portion of the core wire 20 may extend from the distal end 218 of the balloon. The core wire 20 may be provided to confer the desired torquability and pushability to the region of the inflation tube 14 through which it extends. In one aspect, the core wire 20 may be secured to the inflation tube 14 at one or more locations along its length. In other aspects, the core wire 20 may be secured in inflation tube lumen 24 or within other structures independent from inflation tube lumen 24. An atraumatic tip 90 may be secured to the distal end 220 of core wire 20.

### —Overview of Individual Components

In particular, a proximal tube 12 is configured to guide and position portions of the inflation tube 14 within a patient. The proximal tube 12 may function as the point of articulation for a user as the inflation tube 14 is introduced into a bodily lumen of a patient. The proximal lumen 22 of the proximal tube 12 extends over at least a portion of the length of the proximal tube 12. The proximal lumen 22 of the proximal tube 12 may extend longitudinally within the proximal tube 12 from a first proximal tube opening 32 to a second proximal tube opening 42 defined by the proximal tube 12. The proximal lumen 22 is generally configured to receive a fluid, such as, for example, inflation media, and communicate the fluid at least to the second proximal tube opening 32. The proximal tube lumen 22 is typically in fluid communication with the inflation tube lumen 24 of the inflation tube 14.

The proximal tube 12 may further define a proximal tube notch 52 at a distal end 212 of the proximal tube 12. The proximal tube notch 52 may be configured to secure a portion of a core wire 20 extending from an inflation tube 14. The proximal tube notch 52 is generally sized and shaped to receive the proximal end 120 of core wire 20. The proximal tube notch 52 may be defined by one or more notch surfaces 92 of proximal tube 12 at a distal end 212 of the proximal tube 12. The proximal end 120 of the core wire 20 may be secured in the proximal tube notch 52 at the distal end 212 of the proximal tube 12. The proximal tube notch 52 is generally configured to permit fluid to flow through the proximal lumen 22 to or from the second proximal tube opening 42 of the proximal tube 12 when the first end 120 of a core wire 20 is secured within the proximal tube notch 52. The proximal tube notch 52 generally extends from the distal end 212 of the proximal tube 12 to a location along the tube which is proximal to the distal end 212 of the proximal tube 12. The proximal tube notch 52 is generally shaped to receive a core wire mating surface 82, shown in phantom in FIG. 4, at a proximal end 120 of core wire 20.

In one aspect, the width of the proximal tube notch 52 is less than the diameter of the core wire 20. In other aspects, the width of the proximal notch may be the same as or greater than the diameter of the core wire 20. The portion of the proximal tube 12 defining the proximal tube notch 52 may be configured to be secured to the core wire 20. The proximal tube notch 52 generally extends from the distal end 212 of the proximal tube 12 to a location along the proximal tube 12 which is proximal to the distal end 212 of the proximal tube 12. The proximal tube notch 52 may extend into the proximal lumen 22. When the proximal tube 12 is secured to the inflation tube 14, the proximal lumen 22 and the inflation tube lumen 24 together may form a continuous passage extending between a proximal end 112 of proximal tube 12 and a distal inflation opening 44 of inflation tube 14. A bridge tube 30 may be provided between the proximal lumen 22 and inflation tube lumen 24 to assure a continuous passage.

The proximal tube 12 may be made from a variety of materials including polymers, metals, and various composite materials. In one aspect, the proximal tube 12 is made of a stainless steel. In another aspect, the proximal tube 12 is made of nitinol. Typically, the proximal tube 12 is configured to have a desired elastic range. The proximal tube 12 may be configured to have a desired balance of longitudinal stiffness and torsional rigidity based on the characteristics of the inflation tube 14. The longitudinal stiffness, at least in part, dictates the push characteristics for the proximal tube 12. The torsional rigidity, at least in part, dictates the precision of the rotational control provided by the proximal tube 12.

The proximal tube 12 may have various outside diameters 312 and lengths depending on the particular application for the steerable balloon apparatus 10. Generally, the proximal tube 12 is configured to at least support inflation of the associated balloon 18. The particular configuration of proximal tube 12 may depend upon whether or not the proximal tube 12 is intended primarily for use as a balloon catheter or as a wire support for other guidewires or catheters. For use primarily as a balloon catheter, a steerable balloon apparatus 10 may be configured to support larger volumes of fluid than when the steerable balloon apparatus 10 used as a delivery rail for other devices. In such applications for balloon inflation, the proximal tube 12 may have an outside diameter 312 of about 0.024 inches and a lumen diameter of about 0.020 inches. This outside diameter 312 can provide the desired torsional rigidity without being too longitudinally stiff. The inside diameter 412 of the proximal lumen 22 may be selected to provide a desire inflation/deflation time. For intercranial applications where the insertion point is in the femoral artery, the length of the proximal tube 12 can be about 110 centimeters. A proximal tube 12 of this length may keep the proximal tube 12 in the straight portion of the guide. For use of steerable balloon apparatus 10 as a guide wire, the outside diameter 312 can be around 0.014 inches. For other applications requiring access to smaller bodily lumen, an outside diameter 312 of less than 0.014 inches may be used. In one exemplary embodiment, the proximal tube 12 of the steerable balloon apparatus 10 can have an outside diameter 312 of the order of 0.014 inches and a wall thickness of the order of 0.002 inches to maximize the inside diameter 412 of the proximal lumen 22. The proximal tube 12 can be between about 165 cm to about 205 cm in length and although flexible, have a stiffness of about 50-100 N-mm² to impart sufficient lateral stiffness and torque transmission capabilities along its length.

In particular, the inflation tube 14 is configured to facilitate the positioning of the distal end 114 of inflation tube 14 at a desired location within a bodily lumen of a patient and to permit the inflation of the balloon 18. The inflation tube 14 may be used without an associated proximal tube 12 or may be secured to the distal end 214 of a proximal tube 12. When steerable balloon apparatus 10 includes a proximal tube 12, the proximal end 114 of the inflation tube 14 is typically secured to a distal end 212 of the proximal tube 12. In one aspect, the proximal tube 12 may be secured to the inflation tube 14 using apparatus and methods as disclosed in U.S. patent application Ser. No. 11/333,045 entitled Medical Catheters and Methods the disclosure of which is hereby incorporated by reference in its entirety. In another aspect, the inflation tube 14 may be secured to the distal end 212 of the proximal tube 12. If present, a core wire 20 extending from the proximal end 114 of inflation tube 12 may be received within the proximal tube proximal tube notch 52. The core wire 20 may be secured within the proximal tube proximal tube notch 52 and the proximal end 114 of the distal tube 14 may be secured to a portion of the distal end 212 of the proximal tube 12.

The least one inflation tube lumen 24 to permit the communication of fluids along at least a portion of the length of the inflation tube 14. An inner surface 74 of inflation tube 14 defines the inflation tube lumen 24 which typically extends along at least a portion of the length of inflation tube 14. A balloon 18 secured at or near the distal end 214 of the inflation tube 14 is in fluid communication with the inflation tube lumen 24. The inflation tube 14 may further include a core wire 20 extending within the inflation tube 14 over at least a portion of its length. In one aspect, the core wire 20 is positioned within the inflation tube lumen 24 of the inflation tube 14.

The inflation tube lumen 24 of the inflation tube 14 typically extends over at least a portion of the length of the inflation tube 14. The inflation tube lumen 24 is generally configured to communicate a fluid along a portion of the length of the inflation tube 14. In one aspect, the inflation tube lumen 24 may be configured to communicate a fluid from a proximal end 114 to a distal end 114 of the inflation tube 14 or to a location adjacent to the distal end 214 of inflation tube 14. The inflation tube lumen 24 may extend longitudinally within the inflation tube 14 from a proximal inflation opening 34 to a distal inflation opening 44 defined by the inflation tube 14. The proximal inflation opening 34 is typically in communication with the inflation tube lumen 24 at or near the proximal end 114 of the inflation tube 14. The distal inflation tube opening 44 is typically in communication with the inflation tube lumen 24 at or near the distal end 214 of the inflation tube 14. The inflation tube lumen 24 may receive a fluid, such as for example inflation media, through a proximal inflation opening 34 and communicate the fluid to at least the distal inflation tube opening 44. When steerable balloon apparatus 10 includes a proximal tube 12, the inflation tube lumen 24 is typically in fluid communication with the proximal lumen 22 of the proximal tube 12.

The inflation tube 14 may be formed from a single length of tubing or may have a composite structure. When the inflation tube 14 is a composite structure, two or more lengths of tubing may be joined to form the inflation tube 14. As illustrated, a proximal inflation tube 314 may be secured to a distal inflation tube 414. The distal inflation tube 414 may be formed of a reduced diameter and secured within a portion of the inflation lumen 24 defined by the proximal inflation tube portion 314. The distal inflation tube is typically configured to provide the desired flexibility and/or torquability characteristics to the distal end of the inflation tube 14.

The inflation tube 14 may be made from a range of materials and configurations depending upon the intended use for the resultant steerable balloon apparatus 10. In one aspect, the tube may be a metal, such as, for example, stainless steel or nitinol. In another aspect, the inflation tube 14 can be made from one or more polymers such as polyethylene, nylon, polyimide, among others. The materials are generally selected to provide a desired balance of longitudinal stiffness and torsional rigidity based on the characteristics of the inflation tube 14 and, when a core wire 20 is provided, in combination with a core wire 20 extending along at least a portion of the length of the inflation tube 14.

The inflation tube 14 typically has an outside diameter 314 which is the same or smaller than the outside diameter 312 of the proximal tube 12. For use primarily as a balloon catheter, the inflation tube 14 may have an outside diameter 314 of 0.024 inches. The inflation tube lumen 24 may be configured with as large a cross-sectional area as large as possible given the size and particular application for the steerable balloon apparatus 10. In one exemplary embodiment, the inflation tube 14 of a steerable balloon apparatus 10 has a length from about 15 cm to about 25 cm. The inflation tube 14 has an outside diameter 314 of about 0.014 inches and is secured to a proximal tube 12 having the same outside diameter 312. Inflation tube 14 may have a stiffness of about 25-50 N-mm² or less, to impart the desired flexibility to steerable balloon apparatus 10. Additionally, the flexibility of steerable balloon apparatus 10 may be varied by progressively annealing either a portion, for example, only inflation tube 14, or the entire length of steerable balloon apparatus 10.

A bridge tube 30 may extend between the proximal lumen 22 and the inflation tube lumen 24. The bridge tube 30 defines a bridge tube lumen 60. The bridge tube lumen 60 typically communicates fluids between the proximal lumen 22 and the inflation tube lumen 24. In one aspect, the bridge tube 30 may extend between the second proximal tube opening 42 and the proximal inflation tube opening 34. The bridge tube lumen 60 may extend between proximal bridge tube opening 54 at a proximal end 130 of the bridge tube 30 and a distal bridge tube opening 56 at a distal end 230 of the bridge tube 30. The bridge tube 30 typically has a round external cross-sectional shape. However, the bridge tube 30 may have an external cross-sectional shape which corresponds to the shape of the lumen in which it may be received. The bridge tube lumen 60 of the bridge tube 30 generally extends longitudinally within the bridge tube 30 from a first bridge tube opening 54 defined at a proximal end 118 of the bridge tube 30 to a second bridge tube opening 48 defined at a distal end 218 of the bridge tube 30.

Typically, at least a proximal portion of the bridge tube 30 is secured within the proximal lumen 22 through the second proximal tube opening 42 of proximal tube 12 and at least a distal portion of the bridge tube 30 is secured within the inflation tube lumen 24 of inflation tube 14. The bridge tube 30 may be secured within the proximal lumen 22 and inflation tube lumen 24 by welding, adhesive bonding, or by mechanical interaction such as for example being compressionally fitted within proximal lumen 22 and inflation tube lumen 24. When the bridge tube 30 does not sealingly engage the lumen in which it is positioned, such as for example when the external cross-sectional shape of the bridge tube 30 does not correspond to the shape of the proximal lumen 22 or the inflation tube lumen 24, a sealing compound may be applied to seal any gaps. In one aspect, the adhesive compound used to adhesively bond the bridge tube 30 within the proximal lumen 22 and inflation tube lumen 24 may also function as the sealing compound to seal any gaps between the walls of the proximal lumen 22 and inflation tube lumen 24 and the outer surface of the bridge tube 30.

The extendable member 16 is typically an elongated member which is at least extendable distally from the distal end 214 of the inflation tube 14. At least a portion of the extendable member 16 extends through at least a portion of the inflation chamber 28 of balloon 18. The extendable member may be secured to a distal end 218 of the balloon 18. The extendable member 16 may generally extend from the inflation tube 14 in an orientation parallel or coaxial with the longitudinal axis of the inflation tube 14. The extendable member 16 is generally configured to permit the longitudinal movement of a balloon 18 during inflation and/or deflation. In one aspect, the extendable member 16 may include or be formed as a coil 26. The coil 26 may be formed from a metal wire, polymeric strand, other filament, or other material or structure as will be recognized by those skilled in the art. The wires, strands or filaments may have a round, rectangular, square or other cross-sectional shape. The extendable member 16 may further permit the passage of inflation media or other fluids through fenestrations 36 formed in the extendable member 16. When in the form of a coil 26, a single fenestration 36 may be in the form of a spiral defined between the spirally wound the wire or elongated structure forming the coil.

The extendable member 16 may be secured to the distal end 214 of the inflation tube 14 or may be integral with the inflation tube 14. In one aspect, a proximal end 116 of the extendable member 16 is secured to an outer surface 64 at a distal end 214 inflation tube 14. In another aspect, a proximal end 116 of the extendable member 16 is secured within a distal inflation opening 44 of the inflation tube lumen 24. In another aspect, a portion of the proximal end 116 of the extendable member 16 may be embedded within an aspect of the distal end 214 of inflation tube 14 to secure the extendable member 16 to the inflation tube 14. In another aspect, the proximal end 116 of the extendable member 16 may be secured to a surface at the distal end 214 of inflation tube 14 to secure the extendable member 16 to the inflation tube 14. Upon review of the present disclosure, those skilled in the art will recognize additional ways to secure the extendable member 16 to the inflation tube 14 without departing from the scope of the present invention. A portion of the extendable member 16 may be secured to the distal end 218 of the balloon 18 or may be integral with the distal end 218 of balloon 18 such that when the distal end 218 of balloon 18 extends during expansion, the portion of the expandable member 16 proximal to the distal end 218 of the balloon 18 also extends. As illustrated generally throughout the figures for exemplary purposes, the portion of the expandable member 16 secured to the balloon 18 is the distal end 216 of the expandable member. In one aspect, a distal end 216 of the extendable member 16 is secured to an inner surface 74 at a distal end 214 of the balloon 18. In another aspect, a distal end 216 of the extendable member 16 is secured within an inner surface 74 at a distal end 214 of the balloon 18. In another aspect, a distal end 216 of the extendable member 16 is secured to or within an end cap 48 which is secured to a distal end 214 of the balloon 18. In yet another aspect, a portion of the distal end 216 of the extendable member 16 may be embedded within an aspect of the distal end 218 of balloon 18 to secure the extendable member 16 to the balloon 18. Upon review of the present disclosure, those skilled in the art will recognize additional ways to secure the extendable member 16 to the balloon 18 without departing from the scope of the present invention.

The extendable member 16 may be configured to inhibit the deformation of a balloon 18 while positioning the steerable balloon apparatus 10 in a patient. In one aspect, the extendable member 16 may inhibit the deformation of the balloon 18 by inhibiting the rotation of the distal end 218 of balloon 18 relative to the inflation tube 14 about the longitudinal axis of the inflation tube 14. The extendable member 16 may further prevent the deformation of balloons 18 by supporting an inner surface 78 of balloon 18.

The extendable member 16 may be made from a range of materials and configurations depending upon the intended use for the resultant steerable balloon apparatus 10. In one aspect, the extendable member 16 may be a metal, such as, for example, stainless steel or nitinol. In another aspect, the extendable member 16 can be made from one or more polymers such as polyethylene, nylon, polyimide, among others. The materials are generally selected to provide a desired balance of longitudinal stiffness and torsional rigidity based on the characteristics of the extendable member 16 and, when present, in combination with a core wire 20.

The extendable member 16 typically has an outside diameter 316 which is the same or smaller than the outside diameter 316 of the inflation tube 16. Further, the outside diameter 316 of the extendable member 16 may vary along the length of the extendable member 16. In one exemplary embodiment, the extendable member 16 may have a length approximately the same as the balloon 18 to which it is secured. The extendable member 16 may have an outside diameter 316 which is less than an inside diameter of the balloon 18. The extendable member 16 may be of a stiffness which imparts the desired flexibility and/or other characteristics to the balloon 18.

A balloon 18 may be provided at or near the distal end 214 of the inflation tube 14 for inflation within the bodily lumen of a patient. In one aspect, a proximal end 118 of a balloon 18 may be positioned at or near the distal end 214 of the inflation tube 14. The balloon 18 defines an inflation chamber 28 to receive inflation media from the inflation tube lumen 24 of the inflation tube 14. In one aspect, the inflation chamber 28 is in fluid communication with the inflation tube lumen 24. In one aspect, the balloon 18 may be positioned over at least one distal inflation tube opening 44 which is in fluid communication with the inflation tube lumen 24.

The balloon 18 may include a sleeve 38 that can be slidably received over a core wire 20, when present. The sleeve 38 may be integral with the balloon 18 or may be a separate structure secured to the balloon 18. The sleeve 38 may form at least a partial seal which may maintain the seal as the sleeve 38 slides along a core wire 20 to permit the inflation of balloon 18. The sleeve/core wire interaction is typically engineered to prevent medically significant amounts of inflation media from leaking from between the core wire 20 and the sleeve 38. The sleeve 38 defines a sleeve passage 58 to receive a portion of the core wire 20. The core wire 20 may extend from the inflation tube 14 into the inflation chamber 28 of the balloon 18 and through the sleeve passage 58 of the sleeve 38. The sleeve passage 58 typically has a shape which corresponds to the cross-sectional shape of the core wire 20 over the region of the core wire 20 passing through the sleeve 38. As the balloon 18 inflates, sleeve 38 typically slides distally along a portion of core wire 20. For deflation, the inflation tube lumen 24 receives fluid from the balloon 18. As the balloon 18 deflates, the sleeve 38 typically slides proximally along a portion of the core wire 20.

The sleeve 38 may be a separate structure which is secured to the balloon 18 such as the end cap 48 which is illustrated in the Figures for exemplary purposes. As illustrated a distal end 248 of end cap 48 is configured to be relatively atraumatic to a vessel of a patient. When integral, the sleeve 38 may be a thickened or reinforced region of the balloon 18 that resists deformation and leaking upon introduction of inflation media into the expansion chamber 28 and inflation of the balloon 18.

When it is a separate structure, the sleeve 38 can be in the form of an end cap 48. As such, end cap 48 may define a sleeve passage 58. The end cap 48 may be peripherally secured to the balloon 18. The end cap 48 may be generally expandable and elastic, it may be generally rigid, or it may be otherwise configured. However, the sleeve passage 58 defined by the end cap 48 is configured to resist deformation and leaking upon introduction of inflation media into the expansion chamber 28 and inflation of the balloon 18. A lubricious coating 82 may be provided between the sleeve 38 and the core wire 20 to reduce frictional forces between the sleeve 38 and core wire 20 during inflation and deflation as the sleeve 38 slides along the core wire 20. In one aspect, the lubricious coating 82 is provided over at least a portion of the sleeve passage 48. In another aspect, the lubricious coating 82 is provided over at least a portion of the length of the core wire 20.

Depending upon the application for the steerable balloon apparatus 10, the balloon 18 may be configured with a wide range of physical specifications and performance characteristics as will be recognized by those skilled in the art upon review of the present disclosure. In one aspect, the balloon 18 may be either compliant or non-compliant. For various applications, the balloon 18 may be configured and sized to provide the desired inflated diameter and length for a treatment and location. In neurovascular applications, the target vessel diameters may range from as large as 10 to 12 millimeters to as small as 2 to 3 millimeters. The balloon 18 may be configured to circumferentially contact the walls of these vessels and may be provided in a variety of different lengths depending on the treatment and/or purpose of the balloon. In compliant embodiments, the balloon 18 may be made from silicone. For neurovascular applications, silicone may provide additional therapeutic benefits relating to spasms that will be recognized by those skilled in the art upon review of the present disclosure. When silicone is used, the silicone material may have a durometer of about 20 to 30. For neurovascular applications, this may give the steerable balloon apparatus 10 the correct ‘feel’ when the balloon is inflated to a pressure of about 1 atmosphere.

When present, the core wire 20 may be secured within the inflation tube 14 and typically extends over at least a portion of the length of the inflation tube 14. The core wire 20 may confer a desired balance of longitudinal stiffness and torsional rigidity characteristics to the inflation tube 14 through which the core wire 20 extends. Further, the core wire 20 may be secured to or extend into the proximal tube 12 when a proximal tube 12 is included in the steerable balloon apparatus 10. When secured to the proximal tube 12 the core wire 20 may transmit the torquing and pushing of the proximal tube 12 by a user to at least the distal portions of the inflation tube 14. In other aspects, the core wire 20 may be used to, at least in part, secure the inflation tube 14 to the proximal tube 12 of a steerable balloon apparatus 10. When secured along a length of the proximal tube 12, the core wire 20 may confer a desired balance of longitudinal stiffness and torsional rigidity characteristics to the portion of the proximal tube 12 through which the core wire 20 extends.

The core wire 20 may be secured within the inflation lumen 24 of the distal tube 14. In one aspect, the core wire 20 may be secured at one or more discrete locations along the length of the core wire 20. The core wire 20 may be secured one or more discrete locations by introducing an adhesive through one or more transverse passages 40 extending between the outer surface 64 and a portion of inner surface 74 defining lumen 30. In other aspects, the core wire 20 may be rotatably and/or slidably received within the inflation tube lumen 24 of the inflation tube 14.

The core wire 20 is typically a metal wire having a circular transverse cross-section as shown in FIG. 8A for exemplary purposes. The core wire 20 is typically made of a rigid but elastic material. Although the core wire 20 is typically made from stainless steel or nitinol, the core wire 20 may be formed from other metals, polymers or composite materials as will be recognized by those skilled in the art upon review of the present disclosure. The core wire 20 is typically a solid wire, however the core wire 20 may be hollow along at least a portion of its length. The core wire 20 may also be in the form of a wound cable, a braided filament, or otherwise alternatively configured as will be recognized by those skilled in the art upon review of the present disclosure. In other aspects, the core wire 20 may be tapered along the distal portion of the core wire such that the decreasing diameter provides greater flexibility to the region of the core wire 20 extending beyond the distal end 214 of the inflation tube 14.

For intercranial applications, the core wire 20 may be about 40 centimeters long when the insertion point is the femoral artery. In an exemplary embodiment where the proximal tube 12 has an outside diameter 312 of 0.014 inches, the proximal end 116 of the core wire 20 can have a diameter 316 of about 0.009 inches where it attaches to the proximal tube 12. The core wire 20 may include several reductions in outside diameter 316 toward the distal end 216 of core wire 20. In this aspect, the core wire 20 may have a diameter of about 0.004 inches at the distal end 216 of the core wire 20.

An atraumatic tip 90 may be attached to the distal end 114 of the extendable member 16 or the core wire 20. The atraumatic tip 90 generally provides a soft, gentle bumper for the distal end 216 of the extendable member 16 or the core wire 20. The atraumatic tip 90 may include a coil 96. The coil 96 may be about 2 cm long and about 0.014 inches in diameter. The coil 96 can be made of 0.002 inches in diameter radio opaque material, preferably platinum. However, other materials known in the art can be used as well. A shaping ribbon may be positioned within the coil 96. The shaping ribbon is typically constructed from a metal and can serve several important functions. The shaping ribbon may serve as a bendable beam to more easily permit a user to induce a curved shape in the atraumatic tip 90 to direct the steerable balloon apparatus 10 through a bodily lumen of a patient. Further, the shaping ribbon may improve the safety of a steerable balloon apparatus 10 by not allowing the coils 96 of the atraumatic tip 90 to stretch out if a portion of the atraumatic tip 90 becomes lodged or otherwise hung up in the bodily lumen of a patient. The proximal end of the shaping ribbon may be attached to the distal end 216 of the core wire 20 and/or the proximal ends of the coils 96. The distal end of the shaping ribbon may be secured to the distal end of the coils 96. The thickness of the shaping ribbon for intercranial applications is typically about 0.002 inches by 0.004 inches. The shaping ribbon is made from a material having the desired combination of ductility and elasticity. Stainless steel of a proper temper is commonly used to provide these characteristics. The coil 96 may terminate in a rounded cap as to be generally atraumatic to the wall of a bodily lumen.

### Description of Exemplary Illustrated Embodiments

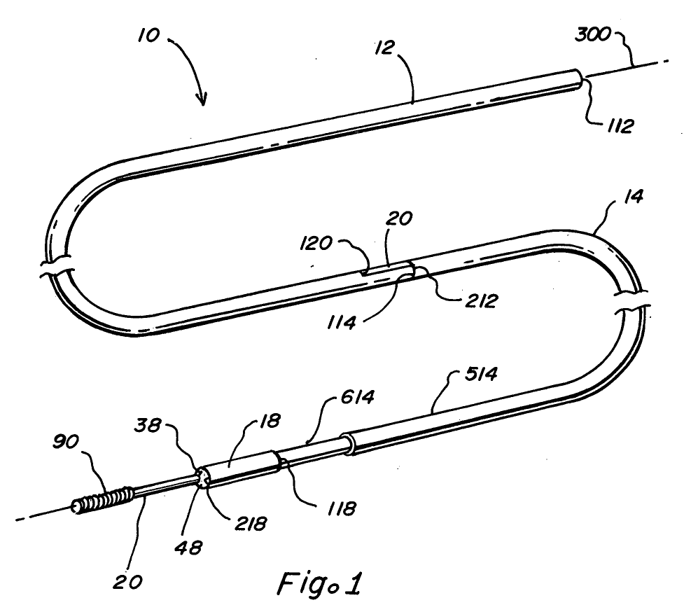

As particularly illustrated for exemplary purposes, FIG. 1 illustrates an embodiment of a steerable balloon apparatus 10 in accordance with the present inventions including both a proximal tube 12, an inflation tube 14 and a balloon 18. FIG. 1 illustrates a perspective view of three portions of a steerable balloon apparatus 10 along the length of the steerable balloon apparatus 10. The proximal tube 12, inflation tube 14, balloon 18 end cap 48, core wire 20 and atraumatic tip 90 are illustrated as having a circular cross-section for exemplary purposes. The illustrated embodiment includes a passage extending from a proximal end 112 of the proximal tube 12 to a distal inflation tube opening 44 underlying the balloon 18 near or at the distal end 214 of the inflation tube 14 to communicate inflation media from the proximal end 112 of the proximal tube 12 to the inflation chamber 28 of the balloon 18. The passage is formed by connecting the proximal tube 12 to the inflation tube 14 such that the proximal lumen 22 of the proximal tube 12 is in fluid communication with the inflation tube lumen 24 of the inflation tube 14.

As illustrated in FIG. 1 for exemplary purposes, the proximal tube 12 is attached to the inflation tube 14 by securing the core wire 20 within a proximal tube notch 52 in the proximal tube 12 and securing the distal end 212 of proximal tube 12 to the proximal end 114 of inflation tube 14. The sleeve 38 is illustrated for exemplary purposes as a end cap 48 defining an axially positioned sleeve passage 58. A portion of the core wire 20 is shown extending through a sleeve passage 58 of sleeve 38. An atraumatic tip 90 is shown secured to the distal end 220 of core wire 20. In the illustrated embodiment, the inflation tube 14 is shown as a composite tube having a proximal inflation tube 314 and a distal inflation tube 414. The proximal inflation tube 314 is illustrated as having a larger diameter than the distal inflation tube 414 for exemplary purposes. Further, the distal inflation tube 414 is secured within an inflation tube lumen 24 of the proximal inflation tube 414 to secure the distal inflation tube 414 to the proximal inflation tube 314 again for exemplary purposes. The inflation tube 14 is generally configured to be directed through a bodily lumen within a patient by a physician manipulating the proximal tube 12 and, once properly positioned, to have the balloon 18 inflated for diagnostic or therapeutic purposes.

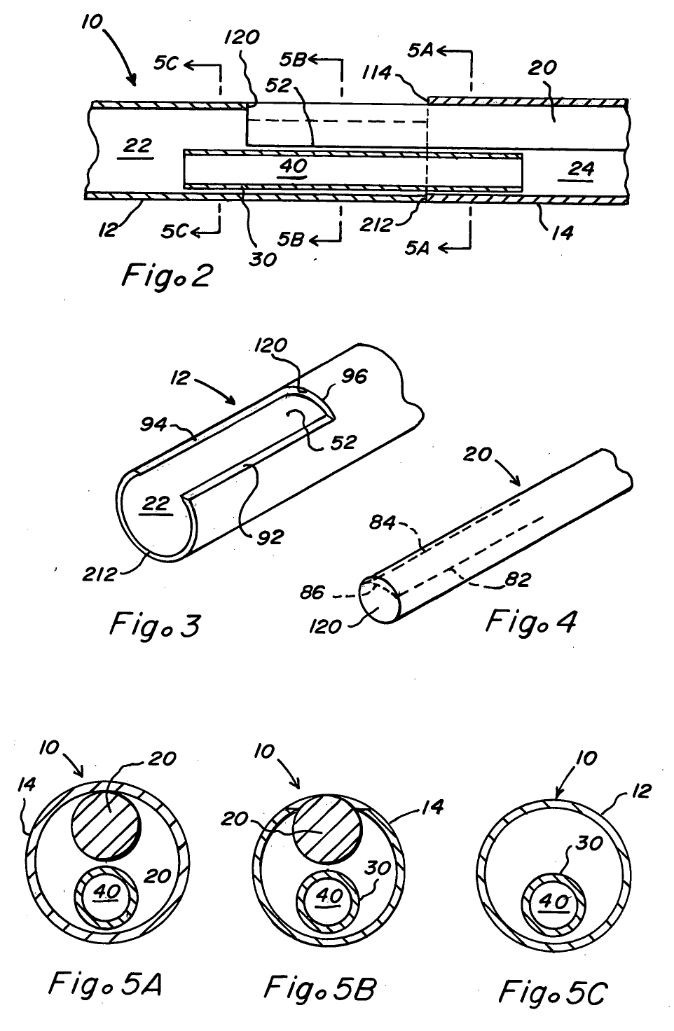

An exemplary junction between a proximal tube 12 and an inflation tube 14 in accordance with the present inventions is illustrated in FIG. 2 with aspects of similar embodiments illustrated in FIGS. 3 to 5C. As illustrated, the inflation tube 14 includes only an inflation tube lumen 24 for exemplary purposes. The inflation tube lumen 24 receives the core wire 20 and carries the fluid along at least a portion of the length of inflation tube 14. The illustrated embodiment further includes a bridge tube 30 extending between the proximal lumen 22 of proximal tube 12 and the inflation tube lumen 24 of the inflation tube 14. The bridge tube 30 is shown extending to a position proximal to the first end 120 of core wire 20. The distal end 212 of the proximal tube 12 is configured to abut the proximal end 114 of the inflation tube 14 when the core wire 20 is positioned within the inflation tube lumen 24 and the core wire 20 is secured within the proximal tube notch 52. The abutting ends 114, 212 may be welded together, adhesively bonded or otherwise secured to one another to seal the proximal lumen 22 and inflation tube lumen 24 about the bridge tube 30. In addition or alternatively to the welded junctions, an adhesive compound and/or a sealing compound may be used to seal the proximal lumen 22 and inflation tube lumen 24 about the bridge tube 30.

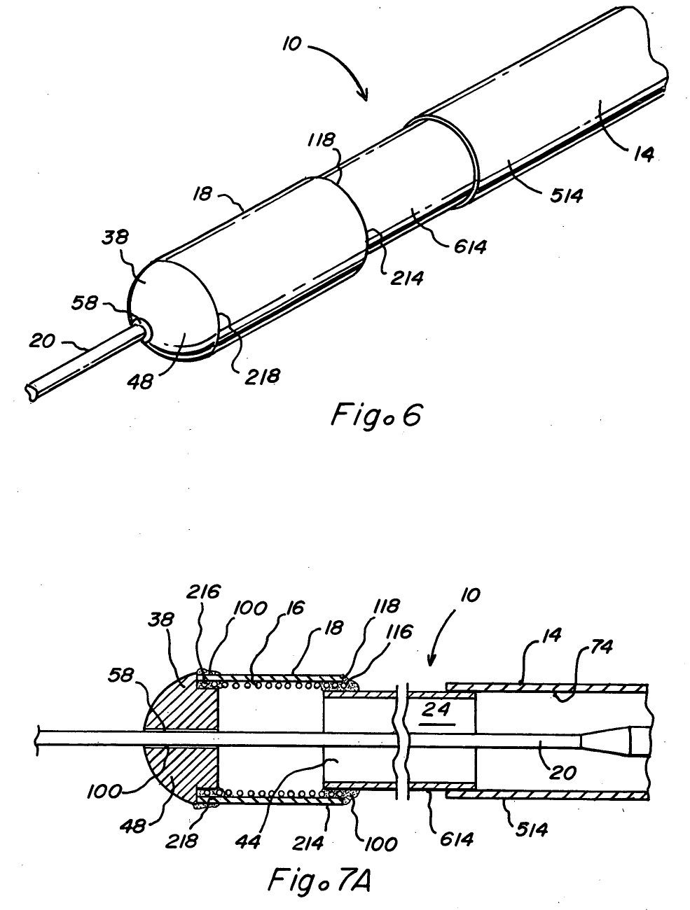

The core wire mating surface 82 at the first end 120 of core wire 20 is secured to the notch surface 92 defining proximal tube notch 52. As shown, core wire mating surface 82 includes a longitudinal mating surface 83 and a perpendicular mating surface 84 which are peripherally secured, as illustrated in FIG. 5B and elsewhere in the Figures, within proximal tube notch 52 of the proximal tube 12 to a longitudinal notch surface 93 and a perpendicular notch surface 94, respectively. The core wire mating surface 82 may be welded, adhesively bonded or otherwise secured to the notch surfaces 92 defining proximal tube notch 52. The bridge tube 30 extends along at least a portion of the length of the core wire 20 from the proximal lumen 22 of the proximal tube 12 into the inflation tube lumen 24 of the inflation tube 14.

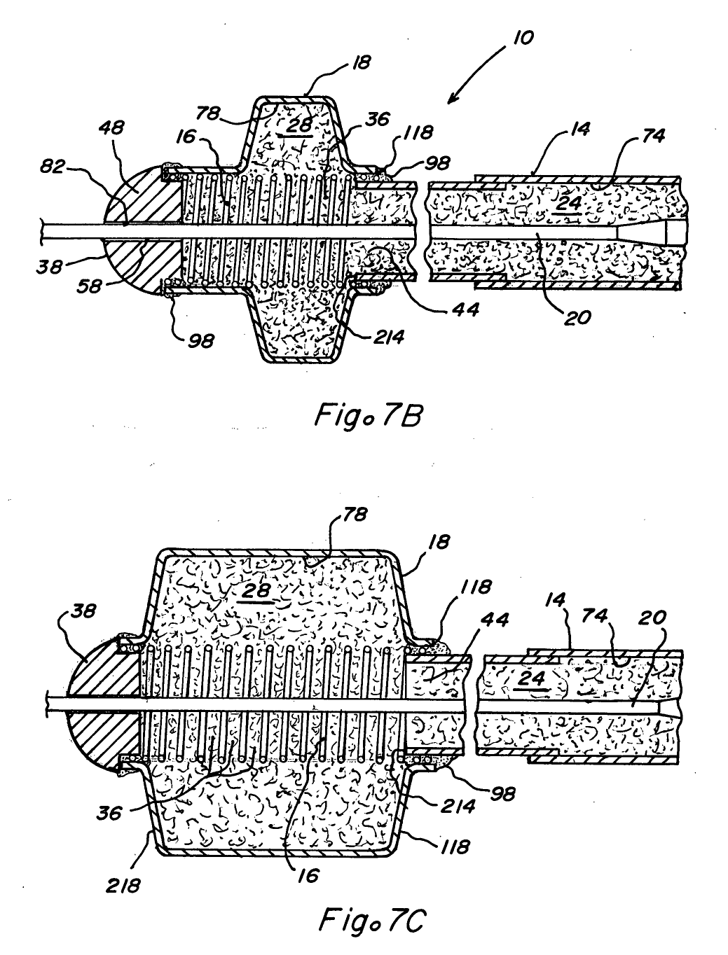

FIG. 6 illustrates the distal portion of an exemplary embodiment of a steerable balloon apparatus 10 in accordance with the present inventions. The illustrated embodiment includes an inflation tube 14, a balloon 18, an end cap 48 and a core wire 20. The inflation tube 14 is illustrated as a composite structure having a proximal inflation tube 514 secured to a distal inflation tube 614. The inflation tube 14 is illustrated as having a circular cross-section for exemplary purposes. The inflation tube includes at least one inflation tube lumen 24 extending from a proximal end 114 of the inflation tube 14 to a distal inflation tube opening 44. The distal inflation tube opening is in communication with the inflation chamber 28 of the balloon 18 at a location at or proximal to the distal end 214 of the inflation tube 14. The inflation lumen 28 is configured to communicate inflation media at least from the proximal portion of the inflation tube 14 to the inflation chamber 28 of the balloon 18. A distal portion of a core wire 20 is shown extending through a sleeve passage 58 of the sleeve 38. The sleeve 38 is shown as a separate component from the balloon 18 and is peripherally secured to balloon 18 for exemplary purposes. The sleeve 38 is particularly illustrated as an end cap 48 defining an axially positioned sleeve passage 58. An atraumatic tip 90 is shown secured to the distal end 220 of core wire 20. In the illustrated embodiment, the inflation tube 14 is generally configured to be directed through a bodily lumen within a patient by a physician manipulating the proximal portion of the inflation tube 14 and, once properly positioned, to have the balloon 18 inflated for diagnostic or therapeutic purposes.

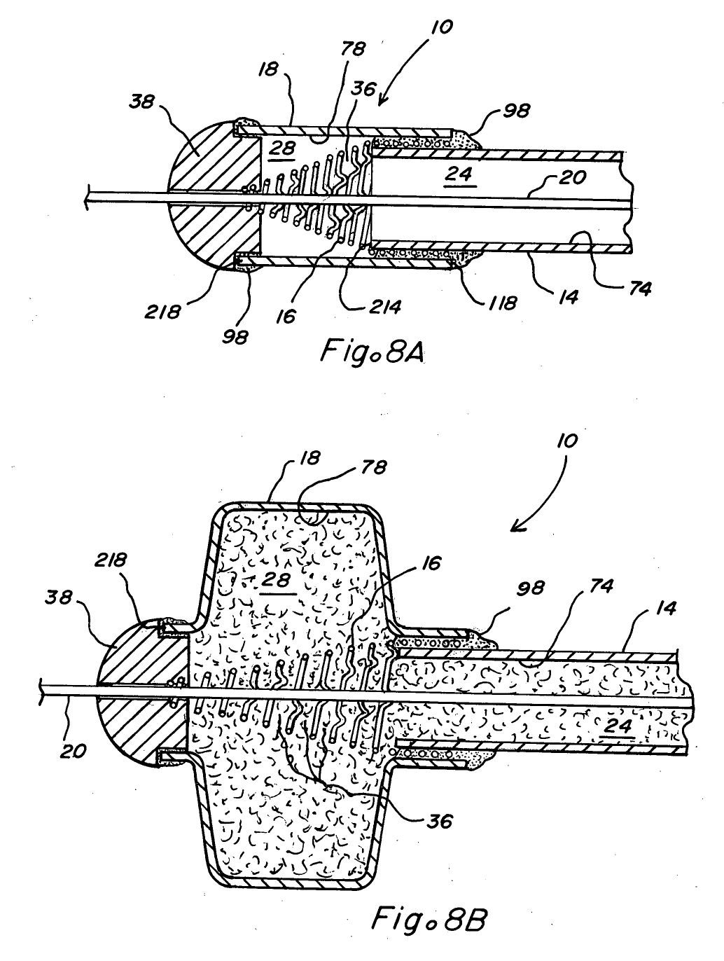

FIGS. 7A to 7C illustrate a cross-section of the distal portion of an exemplary embodiment in accordance with the present invention similar to the embodiment of FIG. 6. FIGS. 7A to 7C show an exemplary cross-section at the distal end 214 of the inflation tube 14 including balloon 18 sequentially expanded from a substantially un-inflated configuration in FIG. 7A to a substantially fully-inflated configuration in FIG. 7C.

FIG. 7A illustrates an exemplary balloon 18 having a proximal end 118 secured over the distal end 214 of the inflation tube 14 with the balloon 18 in a substantially un-inflated configuration. The proximal end 118 of the balloon 18 is in fluid communication with an inflation tube lumen 24 through a of distal inflation tube openings 44 positioned proximal to the distal end 214 of the inflation tube 14. As illustrated, the distal inflation tube opening 44 communicate inflation media into a proximal portion of the inflation chamber 28. To secure a distal end 116 of the extendable member 16 to the balloon 18, the distal end 11 is positioned about a flange at the proximal end 138 of a sleeve 38. As illustrated for exemplary purposes, an adhesive/sealant 98 is used to secure the balloon 18 to the inflation tube 14 and to seal the junction for purposes of balloon inflation. Welding, shrinking, expanding, mechanical bands, or other methods or devices may alternatively be used to secure and/or seal the balloon 18 to the inflation tube 14. The extendable member 16 is secured within the inflation chamber 28. The extendable member is generally configured to support and/or stiffen the balloon 18 while permitting the elongation and contraction of the balloon during the inflation and deflation processes, respectively. The proximal end 116 of the extendable member 16 is secured relative to the distal end 214 of the inflation tube 14. To secure the extendable member 16 to the inflation tube 14, the proximal end 116 is positioned about an outer surface at the distal end 214 of the inflation tube 14. As illustrated for exemplary purposes, an adhesive/sealant 98 is used to secure the extendable member 16 between the balloon 18 and the inflation tube 14. Welding, shrinking, expanding, mechanical bands, or other methods or devices may alternatively be used to secure the extendable member 16 to the inflation tube 16. To secure a distal end 216 of the extendable member 16 to the balloon 18, the distal end 216 of expandable member 16 is positioned about a flange at the proximal end 138 of a sleeve 38. As illustrated for exemplary purposes, an adhesive/sealant 98 is used to secure the extendable member 16 between the balloon 18 and the sleeve 38. Welding, shrinking, expanding, mechanical bands, or other methods or devices may alternatively be used to secure the extendable member 16 to the sleeve 38.

The extendable member 16 is illustrated is illustrated in FIG. 7A as a coil 26 for exemplary purposes. As illustrated, the coil 26 is formed from a spirally wound wire. The coil 26 has a constant diameter along its length. The outside diameter of the coil 26 is shown as substantially the same as the inside diameter of the inner surface 78 of the balloon 18. As such, contact between the inner surface 78 and the coil may support the balloon and prevent deformation as the distal end of the steerable balloon apparatus 10 is positioned within a patient. A single spiraling fenestration 46 is defined by the coil 26. The fenestration 46 may be configured to permit the communication of inflation media.

As illustrated in FIG. 7A, a core wire 20 is secured within the inflation tube 14. The core wire 20 extends distally from the inflation tube 14 through the inflation chamber 28. The core wire 20 is illustrated as substantially coaxial with the extendable member 16. At least a portion of the core wire 20, shown tapered to a reduced diameter for exemplary purposes, extends into and through an inflation chamber 28 defined by the balloon 18 and passes through a sleeve passage 58 of sleeve 38. The sleeve 38 is shown as an end cap 48 peripherally secured to the balloon 18 to form the inflation chamber 28. As illustrated, the sleeve 38 is generally configured to allow the distal end 218 of balloon 18 to slide proximally and distally as the balloon 18 is inflated and deflated respectively. A lubricious coating 82 is provided on the sleeve 38 within the sleeve passage 58 for exemplary purposes. The lubricious coating may alternatively or additionally be provided on an outer surface of the core wire 20.

FIG. 7B illustrates the balloon of FIG. 7A in a partially inflated configuration. The inflation media introduced in the inflation chamber 28 through the inflation lumen 24 and the plurality of distal inflation tube opening 44 is shown first inflating the proximal end 118 of the balloon 18. As the balloon 18 is inflated, the distal end 218 of the balloon is displaced distally along the core wire 20 as the sleeve 38 slides along the core wire 20. The elongated member 16 is extended the longitudinally as the sleeve 38 is displaced by the inflation media. As particularly illustrated, the fenestration 36 widens as the coil 26 that forms the extendable member 16 elongates. FIG. 7C illustrates the balloon 18 of FIGS. 7A and 7B in a fully inflated configuration. The inflation media introduced in the inflation chamber 28 through the inflation lumen 24 and the distal inflation tube opening 44 is shown having inflated the balloon 18 from the proximal end 118 to the distal end 218 of the balloon 18. With the balloon fully inflated, the distal end 218 of the balloon may be displaced distally along the core wire 20 to about the location of atraumatic tip 90 at the distal end 216 of the core wire 20 for exemplary purposes. Further, a substantial portion of the coil 26 is no longer in contact with the inner surface 78 of the balloon 18. As inflation media is removed from the inflation chamber 28, the distal end 218 of the balloon 18 may move proximally along the core wire 20 until the balloon 18 is in a relaxed and deflated condition. In one aspect, the inner surface 78 balloon 18 may again be brought into contact with the coil 26.

FIGS. 8A to 8B illustrate a cross-section of the distal portion of another exemplary embodiment in accordance with the present invention similar to the embodiment of FIG. 6. FIGS. 8A to 8B show an exemplary cross-section at the distal end 214 of the inflation tube 14 including balloon 18 in both a substantially un-inflated configuration in FIG. 8A and a substantially fully-inflated configuration in FIG. 8B.

FIG. 8A illustrates an exemplary balloon 18 having a proximal end 118 secured over the distal end 214 of the inflation tube 14 with the balloon 18 in a substantially un-inflated configuration. The proximal end 118 of the balloon 18 is in fluid communication with an inflation tube lumen 24 through a of distal inflation tube openings 44 positioned proximal to the distal end 214 of the inflation tube 14. As illustrated, the distal inflation tube opening 44 communicate inflation media into a proximal portion of the inflation chamber 28. To secure a distal end 116 of the extendable member 16 to the balloon 18, the distal end 11 is positioned about a flange at the proximal end 138 of a sleeve 38. As illustrated for exemplary purposes, an adhesive/sealant 98 is used to secure the balloon 18 to the inflation tube 14 and to seal the junction for purposes of balloon inflation. Welding, shrinking, expanding, mechanical bands, or other methods or devices may alternatively be used to secure and/or seal the balloon 18 to the inflation tube 14. The extendable member 16 is secured within the inflation chamber 28. The extendable member 18 is illustrated in a conical configuration with the diameter decreasing distally along the extendable member 16. The extendable member 16 is generally configured to permit the elongation and contraction of the balloon 18 during the inflation and deflation processes, respectively while providing the desired flexibility and torquability characteristics to the balloon 28. The proximal end 116 of the extendable member 16 is secured relative to the distal end 214 of the inflation tube 14. To secure the extendable member 16 to the inflation tube 14, the proximal end 116 is again positioned about an outer surface at the distal end 214 of the inflation tube 14 for exemplary purposes. An adhesive/sealant 98 is used to secure the extendable member 16 between the balloon 18 and the inflation tube 14. Welding, shrinking, expanding, mechanical bands, or other methods or devices may alternatively be used to secure the extendable member 16 to the inflation tube 16. To secure a distal end 216 of the extendable member 16 to the balloon 18, the distal end 216 of the extendable member 16 is embedded within at the proximal end 138 of a sleeve 38. Adhesives, welding, expanding, mechanical bands, or other methods or devices may alternatively be used to secure the extendable member 16 to the sleeve 38.

The extendable member 16 is illustrated is illustrated in FIG. 8A as a coil 26 having a decreasing diameter along its length for exemplary purposes. The decreasing diameter may provide torquability, steerability, flexability, stretchability and/or other desired characteristics. As illustrated, the coil 26 is formed from a spirally wound wire. The outside diameter at the proximal end 116 of the coil 26 is shown as substantially the same as the inside diameter of the inner surface 78 of the balloon 18. The inside diameter at the distal end 216 of the coil 26 is sufficient to slidably receive the core wire 20 therethrough. A single spiraling fenestration 46 is defined by the coil 26. Again, the fenestration 46 may be configured to permit the communication of inflation media.

As illustrated in FIG. 8A, a core wire 20 is secured within the inflation tube 14. The core wire 20 extends distally from the inflation tube 14 through the inflation chamber 28. The core wire 20 is illustrated as substantially coaxial with the extendable member 16. At least a portion of the core wire 20, shown tapered to a reduced diameter for exemplary purposes, extends into and through an inflation chamber 28 defined by the balloon 18 and passes through a sleeve passage 58 of sleeve 38. The sleeve 38 is shown as an end cap 48 peripherally secured to the balloon 18 to form the inflation chamber 28. As illustrated, the sleeve 38 is generally configured to allow the distal end 218 of balloon 18 to slide proximally and distally as the balloon 18 is inflated and deflated respectively. A lubricious coating 82 is provided on the sleeve 38 within the sleeve passage 58 for exemplary purposes. The lubricious coating may alternatively or additionally be provided on an outer surface of the core wire 20.

FIG. 8B illustrates the balloon of FIG. 8A in an exemplary fully inflated configuration. The inflation media introduced in the inflation chamber 28 through the inflation lumen 24 and the distal inflation tube opening 44 is shown first inflating the proximal end 118 of the balloon 18. As the balloon 18 is inflated, the distal end 218 of the balloon 18 is displaced distally along the core wire 20 as the sleeve 38 slides along the core wire 20. The elongated member 16 is extended the longitudinally as the sleeve 38 is displaced by the inflation media. As particularly illustrated, the fenestration 36 widens as the coil 26 that forms the extendable member 16 elongates. The inflation media introduced in the inflation chamber 28 through the inflation lumen 24 and the distal inflation tube opening 44 is shown having inflated the balloon 18 from the proximal end 118 to the distal end 218 of the balloon 18. With the balloon 18 fully inflated, the distal end 218 of the balloon 18 has been displaced distally along the core wire 20 to a fully inflated position which may be adjacent to an atraumatic tip 90. As inflation media is removed from the inflation chamber 28, the distal end 218 of the balloon 18 may move proximally along the core wire 20 until the balloon 18 is in a relaxed and deflated condition.

To use a steerable balloon apparatus 10 in accordance with the present invention, a user may insert the distal end of steerable balloon apparatus 10 into a bodily lumen of a patient using, for example, the Seldinger technique. The steerable balloon apparatus 10 is guided through the bodily lumen to a location within the patient requiring treatment. As steerable balloon apparatus 10 is guided through the patient, a user can manipulate the proximal tube 12 or the proximal end 114 of the inflation tube 14 to direct the distal end 214 of the inflation tube 14 through the bodily lumen. When the distal end 214 of the inflation tube 14 is positioned at or near the location within the bodily lumen requiring treatment, the user may initiate the desired treatment. In embodiments where the steerable balloon apparatus 10 includes a balloon 18 at or near the distal end 214 of the inflation tube 14, the balloon 18 may be inflated to a desired size and/or pressure to affect the desired treatment. An steerable balloon apparatus 10 including a balloon 18, properly sized and configured, may enable a user to access more distal or tortuous regions of the body. For example, when the distal portion of the steerable balloon apparatus 10 has an outside diameter of around 0.014 inches, small lumen such as various arteries and veins in the brain and heart may be more easily accessed for diagnosis and/or treatment of the particular lumen or region.

Steerable balloon apparatus 10 may further be used to guide surgical, therapeutic or diagnostic instruments over steerable balloon apparatus 10 to access a desired location in a bodily lumen. When the instrument is positioned at the desired location within the bodily lumen, at least one surgical, therapeutic or diagnostic procedure using the instrument is performed. The instrument may be removed and replaced with a different instrument as required by the treatment, diagnosis, or surgical procedure being performed by the user.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. A steerable balloon apparatus for accessing a bodily lumen of a patient, comprising:
an inflation tube having an outer surface and an inner surface, the inner surface defining an inflation tube lumen, the inflation tube lumen extending along at least a portion of the inflation tube, the inflation tube defining a distal inflation tube opening in communication with the inflation tube lumen;
a balloon defining an inflation chamber, the balloon secured over a distal portion of the inflation tube with the inflation chamber in fluid communication with distal inflation tube opening; and
an extendable member having a proximal end and a distal end, the proximal end of the extendable member secured to a distal end of the inflation tube with at least a portion of the extendable member positioned within the inflation chamber of the balloon, the distal end of the extendable member secured to a distal end of the balloon, the extendable member secured with the distal lumen opening of the lumen, the extendable member extendable between at least a first length and a second length.

2. A steerable balloon apparatus, as in claim 1, further comprising the extendable member defining at least one fenestration.

3. A steerable balloon apparatus, as in claim 1, the extendable member comprising a coil.

4. A steerable balloon apparatus, as in claim 1, further comprising a core wire extending through the inflation tube.

5. A steerable balloon apparatus, as in claim 4, further comprising a core wire extending through the inflation chamber of the balloon.

6. A steerable balloon apparatus, as in claim 5, further comprising the balloon including a sleeve defining a sleeve passage and the core wire slidably received through the sleeve passage.

7. A steerable balloon apparatus, as in claim 6, further comprising the sleeve integral with the balloon.

8. A steerable balloon apparatus, as in claim 7, further comprising a proximal tube defining a proximal lumen extending between a proximal end and a distal end of the proximal tube, the distal end of the proximal tube secured to a proximal end of the inflation tube with the proximal lumen of the proximal tube in fluid communication with the inflation tube lumen of the inflation tube.

9. A steerable balloon apparatus, as in claim 6, further comprising the sleeve secured to the balloon.

10. A steerable balloon apparatus, as in claim 9, the sleeve comprising an end cap peripherally secured to the balloon.

11. A steerable balloon apparatus, as in claim 10, further comprising a proximal tube defining a proximal lumen extending between a proximal end and a distal end of the proximal tube, the distal end of the proximal tube secured to a proximal end of the inflation tube with the proximal lumen of the proximal tube in fluid communication with the inflation tube lumen of the inflation tube.

12. A steerable balloon apparatus, as in claim 4, further comprising a proximal tube defining a proximal lumen extending between a proximal end and a distal end of the proximal tube, the distal end of the proximal tube defining a proximal tube notch, a proximal end of the core wire extending from a proximal end of the inflation tube, the proximal end of the core wire secured within the proximal tube notch of the proximal tube.

13. A steerable balloon apparatus, as in claim 12, further comprising a distal end of the proximal tube secured to a proximal end of the inflation tube.

14. A steerable balloon apparatus, as in claim 12, further comprising a longitudinal core wire mating surface of the core wire secured to a longitudinal notch surface defining a portion of the proximal tube notch.

15. A steerable balloon apparatus, as in claim 12, further comprising a perpendicular core wire mating surface of the core wire secured to a perpendicular notch surface of the proximal tube.

16. A steerable balloon apparatus, as in claim 15, further comprising a longitudinal core wire mating surface of the core wire secured to a longitudinal notch surface defining a portion of the proximal tube notch.
